(19) United States
(12) Patent Application Publication
(10) Pub. No.: US 2018/0263552 A1
(43) Pub. Date: Sep. 20, 2018

(54) BIOMETRIC AND LOCATION BASED SYSTEM AND METHOD FOR FITNESS TRAINING

(71) Applicant: Charge LLC, Manhattan, IL (US)

(72) Inventors: Rory Justin Graman, Virginia Beach, VA (US); Matthew Thomas Knippen, Manhattan, IL (US); Kyle Edward Markham, Chicago, IL (US)

(73) Assignee: Charge LLC, Manhattan, IL (US)

(21) Appl. No.: 15/923,618

(22) Filed: Mar. 16, 2018

Related U.S. Application Data
(60) Provisional application No. 62/472,966, filed on Mar. 17, 2017.

(51) Int. Cl. A61B 5/00; A61B 5/0205

(52) U.S. Cl. CPC A61B 5/486; A61B 5/02055; A61B 5/02438; A61B 5/0022; A61B 5/7465

(57) ABSTRACT

A system and method to send biometric and location-based data to provide live remote fitness advice. The systems allows fitness trainers to receive live biometric and location-based data from the athletes, wherever they are training, in order to improve the fitness performance of the athletes being trained, while they are engaged in a training.

### CROSS-REFERENCE TO RELATED APPLICATION

This application claims the benefit of priority of U.S. provisional application No. 62/472,966, filed Mar. 17, 2017, the contents of which are herein incorporated by reference.

### BACKGROUND OF THE INVENTION

The present invention relates to fitness training, and more particularly to facilitated fitness training with a fitness trainer.

Streaming fitness is a current trend, but there is presently no method or process that allows for remote personal interaction between a fitness trainer and an athlete based on the athletes live biometric or location-based data. While there are methods for such training interaction when the trainer and athlete being co-located, there are currently no methods or process that makes this possible irrespective of either of the individual's locations.

The problem with the other systems in the field of streaming fitness is that there is no way for fitness trainers to receive live biometric and location-based data from the athletes that they are training in order to provide fitness feedback based on this data live and in real time. This means that fitness trainers that are streaming fitness advice have no accurate or meaningful way to provide effective feedback in order to improve the fitness performance of athletes since they do not have access to the athlete's live biometric and location-based data.

As can be seen, there is a need for an improved system and method that allows fitness trainers to remotely receive live biometric and location-based data from the athletes they are training in order to improve the fitness performance of the athletes being trained, while they are being trained.

### SUMMARY OF THE INVENTION

In one aspect of the present invention, a fitness training system for remote monitoring and analysis of an athlete's biometric and location based performance metrics is disclosed. The fitness training system includes a fitness monitoring device carrying one or more biometric sensors configured to monitor a biometric signal of the athlete during the athlete's performance of a fitness training regimen. A first computing device is maintained in close proximity to the athlete during the athlete's performance of the fitness training regimen. The first computing device is configured for communication with the fitness monitoring device. The first computing device is configured to receive biometric output data corresponding to the biometric signal of the athlete during the athlete's performance of the fitness training regimen. A second computing device, associated with a fitness trainer, is configured to receive the biometric output data from the first computing device during the athlete's performance of the fitness training regimen. The second computing device also receives a geolocation coordinate from one of the fitness monitoring device or the first computing device, where the second computing device is located remotely from the first computing device.

In other aspects of the invention, the one or more biometric sensors includes a sensor selected from the group consisting of: a heart rate sensor, a respiratory rate sensor, a temperature sensor, a blood pressure sensor, a blood oxygen level sensor, and a breath carbon dioxide sensor.

The fitness training system may also include a feedback channel established between the second computing device and the first computing device, wherein the feedback channel is configured to communicate one or more of a text message, a cellular network phone call, a VOIP call, or a video chat session between the fitness trainer and the athlete during performance of the training regimen.

In some embodiments, the geolocation coordinate corresponds to a global positioning system (GPS) receiver embedded with the fitness monitoring device. In other embodiments, the geolocation coordinate corresponds to a global positioning system (GPS) receiver embedded with the first computing device.

Other aspects of the invention include a method of fitness training providing remote monitoring and analysis of an athlete's biometric and location based performance metrics of claim. The method includes operating a fitness monitoring device carrying one or more biometric sensors configured to monitor a biometric signal of the athlete during the athlete's performance of a fitness training regimen. A first computing device maintained in close proximity to the athlete during the athlete's performance of the fitness training regimen, receives biometric output data corresponding to the biometric signal of the athlete during the athlete's performance of the fitness training regimen. A second computing device associated with a fitness trainer, receives the biometric output data from the first computing device during the athlete's performance of the fitness training regimen and a geolocation coordinate from one of the fitness monitoring device or the first computing device.

In some embodiments, the one or more biometric sensors include a sensor selected from the group consisting of: a heart rate sensor, a respiratory rate sensor, a temperature sensor, a blood pressure sensor, a blood oxygen level sensor, a breath carbon dioxide sensor, an accelerometer and a gyroscope. The geolocation coordinate may correspond to a global positioning system (GPS) receiver embedded with the fitness monitoring device. Alternatively, the geolocation coordinate may correspond to a global positioning system (GPS) receiver embedded with the second computing device.

The biometric output data and the geolocation coordinate data are analyzed by the fitness trainer to determine the physiologic performance of the athlete. The athlete may then receive a feedback instruction from the fitness trainer via a feedback channel established between the second computing device and the first computing device, wherein the feedback channel is configured to communicate the feedback instruction via one or more of a text message, a cellular network phone call, a VOIP call, or a video chat session.

These and other features, aspects and advantages of the present invention will become better understood with reference to the following drawings, description and claims.

### BRIEF DESCRIPTION OF THE DRAWINGS

FIG. 1 is a schematic diagram of a training system architecture according to aspects of the invention.

FIG. 2 is a flowchart illustrating a monitoring and process according to aspects of the invention.

FIG. 3 is another flowchart illustrating a fitness trainer's analysis and feedback for an athlete performing a fitness regimen.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, embodiments of the present invention provides a system and method for providing remote personal fitness training for an athlete based on biometric and location based data for the athlete's training regimen.

The present invention allows an athlete's live biometric and location-based data to be communicated to and received by a fitness trainer. The fitness trainer is then able to immediately communicate fitness advice to the athlete based on the athlete's live biometric and location-based data. In practicing the invention, it does not matter where the fitness trainer or athlete are located, thus allowing for live, personal interaction from athletes with fitness trainers and from fitness trainers to athletes live and irrespective of location for the purpose of fitness improvement.

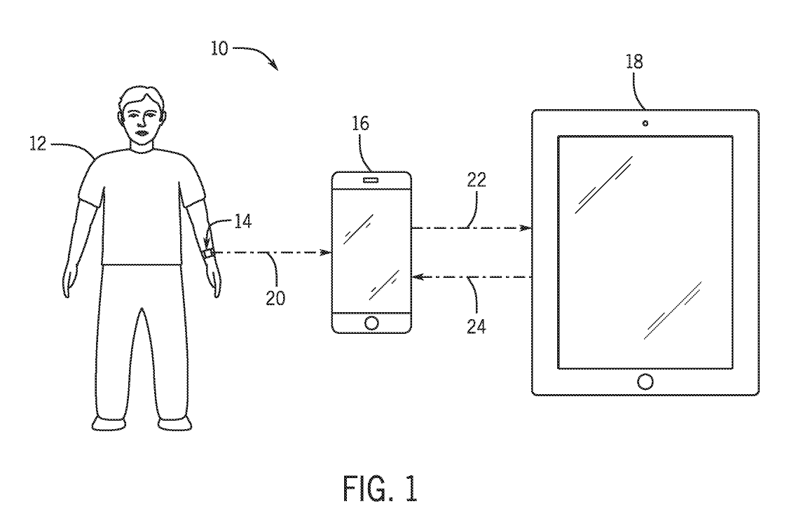

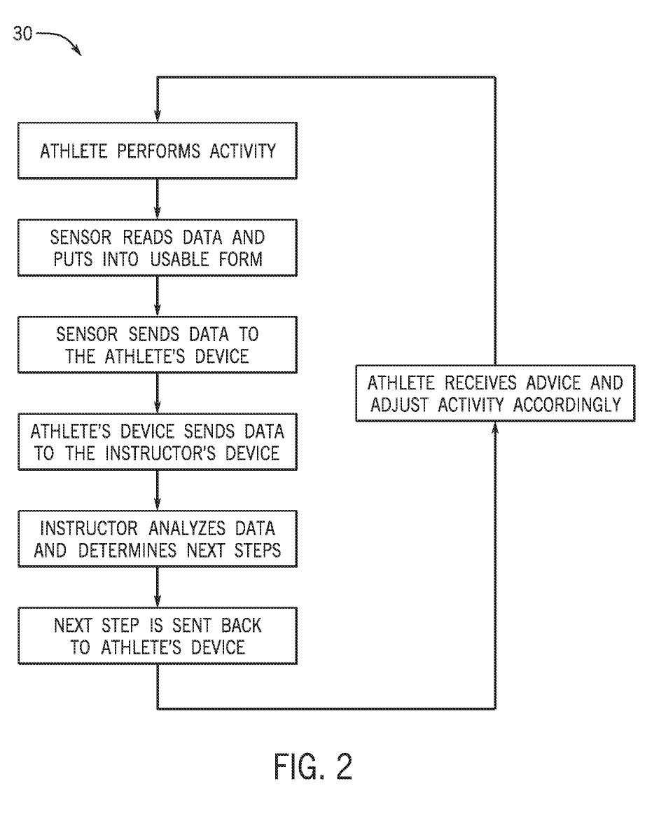

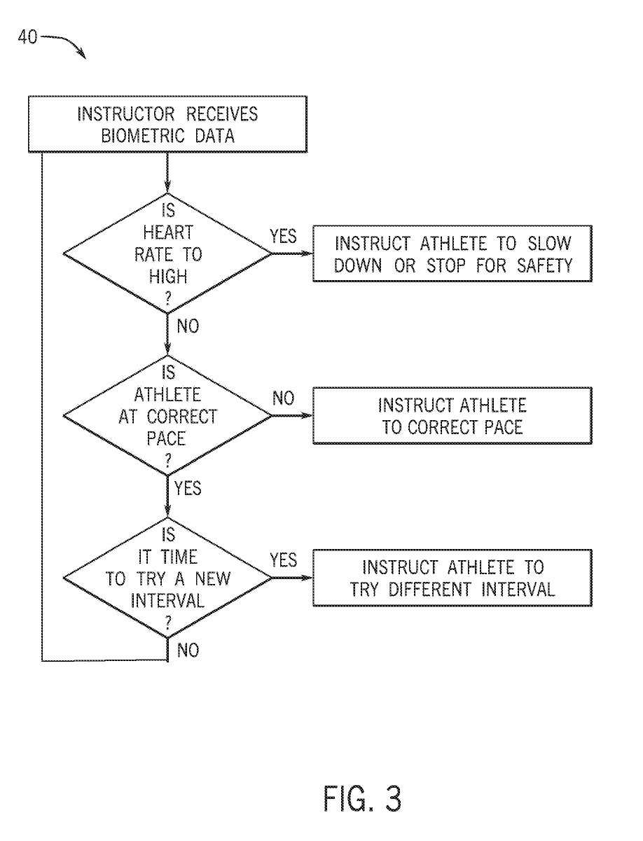

As seen in reference to FIG. 1a-3, an athlete 12 may be engaged in one of a number of fitness activities 1, such as running, walking, swimming, bike riding or waterskiing, and the like. The athlete 12 is equipped with one or more sensors 14 configured to monitor a biometric output signal of the athlete 12. By way of non-limiting example, the sensor 14 may include one or more of a heart rate sensor, a respiratory rate sensor, a temperature sensor, a blood pressure sensor, a blood oxygen level sensor, and a breath carbon dioxide sensor to measure an athlete's physiologic condition. The biometric sensor 14 may also be configured to measure a performance level of an athlete's of physical activity. These additional biometric sensors 14 may include an accelerometer, a barometer, and the like to determine parameters such as a distance traveled, a pace, a current trajectory, or a number of flights of stairs climbed.

The biometric sensors 14 may be integrated into a wearable device worn by the athlete. The biometric sensors 14 are configured to communicate biometric and location based data with a mobile computing device 16, such as a smart phone, tablet and the like. The communication channel 20 may be a Bluetooth, a Wi-Fi, or a hard-wired connection. Mobile computing device 16 may be worn, carried by or near the athlete 12 during performance of a fitness activity. In some embodiments of the invention, one or more of the biometric sensors 14 may instead be integrated with the mobile computing device 16. Likewise, the mobile computing device 16 may including location determining capabilities, such as a global positioning system (GPS).

The athlete's computing device 16 then communicates the biometric and location based data via a second communications channel 22, which may include communications via the Internet, such as a TCP, UDP, HTTP or similar connection, which may similarly be communicated to a computing device 18 of a personal fitness trainer that may be located remotely from the athlete 12. The fitness trainer is then able to receive the biometric and location-based data concerning the athlete's performance of the fitness activity. The fitness trainer may then assess the biometric and location based data for the athlete's performance and provide advice, guidance, a next course of action, or a helpful/motivational comment via the computing device 18 and the athlete's computing device 16. The fitness trainer's device 18 is configured to communicate 24 the advice, guidance, next course of action, or helpful/motivational comment from the fitness trainer's device 18 to the athlete's device 16, such as via a text message, a cellular network phone call, a VOIP or a video chat session. The athlete 12 can receive the advice, guidance, next course of action, or helpful/motivational comment 10 from their device 16, and update their performance of the fitness activity accordingly.

By following the above-listed steps, a fitness trainer can remotely train an athlete based on the athlete's biometric and location-based data. Once the fitness instructor's device 18 receives the biometric and location based data, the fitness instructor has multiple steps of logic he must then determine. For example, in process 30, if the athlete's heart rate is elevated above an appropriate determined threshold, the instructor can instruct the athlete to slow down or stop performing the fitness activity to avoid injury. Likewise, if the athlete's performance and biometric data indicate that the athlete is not performing to a determined level, the fitness trainer can provide feedback for the athlete to increase their level of activity or performance.

As another non-limiting example, the fitness instructor may instruct that the athlete 12 perform a fitness activity such as running. The instructor may provide goal parameters such as perform a sprint at a seven-minute pace. When the fitness instructor receives the biometric and location based data 5 and determines that the athlete 12 is currently running at only a seven-and-a-half minute pace, he/she could offer a helpful/motivational comment 10 for him/her to increase his current pace.

Alternatively, systems could also be utilized in an instructional capacity. The fitness trainer could perform the fitness activity and provide their biometric and location-based data from a sensor and communicate it through the same steps listed above to the athlete 12. This would allow the athletes 2 to compare themselves and/or compete with the fitness trainer.

In yet another embodiment, the fitness trainer may give the advice, guidance, a next course of action, or helpful/motivational comment 10 to the athlete 12 while performing the fitness activity. This could also be used to give the athlete 12 the advice, guidance, a next course of action, or helpful/motivational comment after the athlete 12 has completed the fitness activity.

The present invention can be used by a physical therapist, physician, or other medical professional in order to receive live biometric or location-based data from their patient. The physical therapist, physician, or other medical professional can then provide medical advice to their patient or use this data to decide whether or not to engage emergency services for their patient.

The system of the present invention may include at least one computer with a user interface. The computer may include any computer including, but not limited to, a desktop, laptop, and smart device, such as, a tablet and smart phone. The computer includes a program product including a machine-readable program code for causing, when executed, the computer to perform steps. The program product may include software which may either be loaded onto the computer or accessed by the computer. The loaded software may include an application on a smart device. The software may be accessed by the computer using a web browser. The computer may access the software via the web browser using the internet, extranet, intranet, host server, internet cloud and the like.

The computer-based data processing system and method described above is for purposes of example only, and may be implemented in any type of computer system or programming or processing environment, or in a computer program, alone or in conjunction with hardware. The present invention may also be implemented in software stored on a non-transitory computer-readable medium and executed as a computer program on a general purpose or special purpose computer. For clarity, only those aspects of the system germane to the invention are described, and product details well known in the art are omitted. For the same reason, the computer hardware is not described in further detail. It should thus be understood that the invention is not limited to any specific computer language, program, or computer. It is further contemplated that the present invention may be run on a stand-alone computer system, or may be run from a server computer system that can be accessed by a plurality of client computer systems interconnected over an intranet network, or that is accessible to clients over the Internet. In addition, many embodiments of the present invention have application to a wide range of industries. To the extent, the present application discloses a system, the method implemented by that system, as well as software stored on a computer-readable medium and executed as a computer program to perform the method on a general purpose or special purpose computer, are within the scope of the present invention. Further, to the extent, the present application discloses a method, a system of apparatuses configured to implement the method are within the scope of the present invention.

It should be understood, of course, that the foregoing relates to exemplary embodiments of the invention and that modifications may be made without departing from the spirit and scope of the invention as set forth in the following claims.

What is claimed is:

1. A fitness training system for remote monitoring and analysis of an athlete's biometric and location based performance metrics, comprising:
a fitness monitoring device carrying one or more biometric sensors configured to monitor a biometric signal of the athlete during the athlete's performance of a fitness training regimen;
a first computing device maintained in close proximity to the athlete during the athlete's performance of the fitness training regimen, the first computing device in communication with the fitness monitoring device, the first computing device configured to receive biometric output data corresponding to the biometric signal of the athlete during the athlete's performance of the fitness training regimen;
a second computing device associated with a fitness trainer, the second computing device configured to receive the biometric output data from the first computing device during the athlete's performance of the fitness training regimen and a geolocation coordinate from one of the fitness monitoring device or the first computing device, where the second computing device is located remotely from the first computing device.

2. The fitness training system for remote monitoring and analysis of an athlete's biometric and location based performance metrics of claim 1, wherein the one or more biometric sensors include a sensor selected from the group consisting of: a heart rate sensor, a respiratory rate sensor, a temperature sensor, a blood pressure sensor, a blood oxygen level sensor, a breath carbon dioxide sensor, an accelerometer and a gyroscope.

3. The fitness training system for remote monitoring and analysis of an athlete's biometric and location based performance metrics of claim 2, further comprising:
a feedback channel established between the second computing device and the first computing device, wherein the feedback channel is configured to communicate one or more of a text message, a cellular network phone call, a VOIP call, or a video chat session between the fitness trainer and the athlete during performance of the fitness training regimen.

4. The fitness training system for remote monitoring and analysis of an athlete's biometric and location based performance metrics of claim 3, wherein the geolocation coordinate corresponds to a global positioning system (GPS) receiver embedded with the fitness monitoring device.

5. The fitness training system for remote monitoring and analysis of an athlete's biometric and location based performance metrics of claim 3, wherein the geolocation coordinate corresponds to a global positioning system (GPS) receiver embedded with the first computing device.

6. A fitness training method for providing remote monitoring and analysis of an athlete's biometric and location based performance metrics of claim, comprising:
operating a fitness monitoring device carrying one or more biometric sensors configured to monitor a biometric signal of the athlete during the athlete's performance of a fitness training regimen;
receiving, on a first computing device maintained in close proximity to the athlete during the athlete's performance of the fitness training regimen, biometric output data corresponding to the biometric signal of the athlete during the athlete's performance of the fitness training regimen;
receiving, on a second computing device associated with a fitness trainer, the biometric output data from the first computing device during the athlete's performance of the fitness training regimen and a geolocation coordinate from one of the fitness monitoring device or the first computing device.

7. The fitness training method for providing remote monitoring and analysis of an athlete's biometric and location based performance metrics of claim 6, wherein the one or more biometric sensors include a sensor selected from the group consisting of: a heart rate sensor, a respiratory rate sensor, a temperature sensor, a blood pressure sensor, a blood oxygen level sensor, and a breath carbon dioxide sensor.

8. The fitness training method for providing remote monitoring and analysis of an athlete's biometric and location based performance metrics of claim 6, wherein the geolocation coordinate corresponds to a global positioning system (GPS) receiver embedded with the fitness monitoring device.

9. The fitness training method for providing remote monitoring and analysis of an athlete's biometric and location based performance metrics of claim 8, further comprising:
analyzing the biometric output data and the geolocation coordinate data to determine the physiologic performance of the athlete.

10. The fitness training method for providing remote monitoring and analysis of an athlete's biometric and location based performance metrics of claim 9, further comprising:
receiving a feedback instruction from the fitness trainer via a feedback channel established between the second computing device and the first computing device, wherein the feedback channel is configured to communicate the feedback instruction via one or more of a text message, a cellular network phone call, a VOIP call, or a video chat session.

11. The fitness training method for providing remote monitoring and analysis of an athlete's biometric and location based performance metrics of claim 6, wherein the geolocation coordinate corresponds to a global positioning system (GPS) receiver embedded with the first computing device.
